(19) United States
(12) Patent Application Publication
(10) Pub. No.: US 2002/0147363 A1
(43) Pub. Date: Oct. 10, 2002

(54) Method and apparatus for rapid screening of multiphase reactant systems

(75) Inventors: James Lawrence Spivack, Cobleshill, NY (US); Bruce Fletcher Johnson, Scotia, NY (US); John Yaw Ofori, Niskayuna, NY (US); Eric Douglas Williams, Schenectady, NY (US)

Correspondence Address: GENERAL ELECTRIC COMPANY, CRD PATENT DOCKET ROOM 4A59, P O BOX 8, BUILDING K 1 SALAMONE, SCHENECTADY, NY, 12301, US

(73) Assignee: General Electric Company

(21) Appl. No.: 10/109,470

(22) Filed: Mar. 28, 2002

Related U.S. Application Data
(63) Continuation-in-part of application No. 09/345,539, filed on Jun. 30, 1999.

(51) Int. Cl. C07C 51/15

(52) U.S. Cl. 562/424

(57) ABSTRACT

In one embodiment, the present invention provides a method of producing a homogeneous chemical reaction utilizing multiphase starting materials. The method includes the steps of providing a first reactant system embodied in a liquid and contacting the liquid with a second reactant system embodied in a gas. The liquid is arrayed in a form having dimensions such that the reaction rate of the homogeneous chemical reaction is essentially independent of the mass transport rate of the second reactant system into the liquid. The present invention further provides a method of performing simultaneous homogeneous chemical reactions utilizing multiphase reactant systems. The present invention is also directed to vessels for accommodating homogeneous chemical reactions.

### RELATED APPLICATIONS

This application is a continuation in part of U.S. application Ser. No.09/345,539, filed Jun. 30, 1999.

### BACKGROUND OF THE INVENTION

The present invention is directed to a method and apparatus for rapid screening of potentially mass transport limited reactions and, more specifically, to a method and apparatus for running multiple homogeneous reactions in parallel using multiphase reactant systems.

The general evaluation of potential reactants and catalyst systems requires that each potential combination be subjected to reaction conditions that permit the appropriate reaction(s) to take place and that the products of the reaction(s) be determinable at a level that allows discrimination among the potential combinations under conditions that would provide a meaningful correlation to performance in a production scale reactor. These requirements present unique issues in applying combinatorial techniques to multiphase reactant systems. Specifically, in multiphase reactant systems, mass transport often plays a significant role in reaction kinetics or is rate limiting, thereby requiring mechanical mixing of the phases. Therefore, although running multiple simultaneous reactions would be desirable, the screening of potential reactants and catalysts for such systems has traditionally been carried out one experiment at a time.

When some reagents are in a liquid phase and others in a gas phase, traditional chemical engineering practice demands that the two phases be well mixed during the reaction, typically by rapid stirring, sparging, and the like. At production scale, the reaction is typically carried out in a continuous flow reactor. However, the expense involved in constructing and operating production scale continuous reactors has led to the general practice of screening multiphase reactant systems in batch mode. A continuous reactor differs from batch mode in that in the continuous reactor a compositional steady state mixture is typically obtained containing product, starting materials, by-products, fresh and degraded catalysts, and the like. Traditional batch mode reactors have incorporated rapid stirring or gas sparging to facilitate mixing of the phases, which can present difficulties in creating methods which permit running multiple simultaneous reactions. An effective combinatorial model would be capable of discriminating among potential reactants and catalyst systems under conditions that would provide a meaningful correlation to performance in a continuous flow reactor. However, the aforementioned mass transport considerations have limited the application of combinatorial techniques to multiphase systems.

As the demand for high performance materials has continued to grow, new and improved methods of providing products more economically are needed to supply the market. In this context, various reactant and catalyst combinations are constantly being evaluated; however, the identities of chemically or economically superior reactant systems for multiphase processes continue to challenge the industry. New and improved methods and devices are needed for rapid screening of multiphase reactant systems.

### BRIEF SUMMARY OF THE INVENTION

The present invention is directed to a method of performing a homogeneous chemical reaction utilizing multiphase reactant systems, said method comprising the steps of:

providing a first reactant system embodied in a liquid;

contacting the liquid with a second reactant system embodied in a gas, the second reactant system having a mass transport rate into the liquid;

wherein the liquid is arrayed in a form having dimensions such that the reaction rate of the homogeneous chemical reaction is essentially independent of the mass transport rate of the second reactant system into the liquid.

The present invention further relates to a method of performing simultaneous homogeneous chemical reactions utilizing multiphase reactant systems. Additionally, the present invention relates to a vessel for carrying out homogeneous chemical reactions utilizing multiphase reactant systems. Finally, the present invention relates to a combinatorial microreactor for carrying out simultaneous homogeneous chemical reactions utilizing multiphase reactant systems.

### BRIEF DESCRIPTION OF THE DRAWINGS

Various features, aspects, and advantages of the present invention will become more apparent with reference to the following description, appended claims, and accompanying drawings, wherein FIG. 1 is a side view of an aspect of an embodiment of the present invention.

FIG. 2 is a partial perspective view of an aspect of an embodiment of the present invention.

FIG. 3 is a side view of an aspect of an alternative embodiment of the present invention.

### DETAILED DESCRIPTION OF THE INVENTION

Terms used herein are employed in their accepted sense or are defined. In this context, the present invention is directed to a method and apparatus for rapid screening of multiphase reactant systems.

As used herein, the expression “between 0 and 5” indicates a range of numbers bounded by the numbers 0 and 5, said range not including the numbers 0 and 5.

Unless otherwise noted, the term “reactant system” can include reactants, solvents, carriers, catalysts, and chemically inert substances that are present to affect a physical property of one or more components of the reactant system. In this regard, the liquid solution of the first reactant system may include a solvent which itself undergoes a chemical reaction upon contact with said second reaction system. The homogenous gaseous second reactant system may include a plurality of gaseous components, at least one of said gaseous components undergoing chemical reaction when contacted with said first reaction system. In alternative embodiments, the first reactant system is dissolved in a solvent to afford a liquid solution, said solvent not being included in said first reactant system.

As used herein the term “embodied” means to be “dissolved in” and includes the situation in which a reactant system is dissolved in an inert liquid or gas and the situation in which the liquid or gas itself forms part of the reactant system.

Contact between the first reactant system in solution and the homogeneous gaseous reactant system is effected in a reaction vessel, the solution being arrayed within the reaction vessel in such a manner such that the reaction rate between the first reactant system in solution with the gaseous second reactant system is independent of the mass transport rate of the gaseous reactant system into the solution. In describing the first reactant system in solution as being “arrayed within the reaction vessel” it is meant that the solution of the first reactant system is deposited in the reaction vessel as a film, layer, droplet, bead, strand, ring, or like array, and is not subjected to mechanical agitation during the reaction. Regardless of the form, for example a film, layer, droplet, bead, strand, ring, or the like, in which the solution of the first reactant system is deposited in the reaction vessel, the liquid should have the characteristic that the reaction rate between the first reactant system in solution and the gaseous second reaction system is essentially independent of the mass transport rate of the second reactant system into the solution. Typically, this characteristic will depend upon the dimensions of the film, layer, droplet, bead, strand, ring, or like form of the solution of the first reactant system arrayed within the reaction vessel.

As noted, the liquid is arrayed in a form having dimensions such that rate of reaction between the first reactant system and the second reactant system is “essentially independent” of the mass transport rate of the second reactant system into the liquid. In this context, “essentially independent” means that in comparison with other possible rate limiting factors, mass transport limitations are sufficiently low to allow comparative evaluations of potential reactant system components. This allows one to compare the performance of one first reaction system with another and in so doing allows the identification of superior catalyst systems comprised by the various first reactant systems undergoing evaluation. The optimum form of the arrayed liquid, for example a film, layer, droplet, bead, strand, ring, or like array, and the dimensions of the arrayed liquid can vary based on reaction conditions and the identity of reactant system components. Those skilled in the art will readily realize that in various systems, some minimum dimensions of the arrayed liquid may be required to overcome the effects of evaporation or the formation of micro amounts of precipitate and the like.

The method of the present invention requires that the rate of reaction between the first reactant system and the second reactant system be essentially independent of the mass transport rate of the second reactant system into the liquid. In order to determine that the liquid has been arrayed in a form having dimensions such that this condition is met may be readily determined as follows. First, known but varying amounts, for example 10 to 100 milligrams, of the liquid comprising the first reactant system are arrayed in a single form (film, layer, droplet, bead, strand, ring, or like form) in a series of identical reaction vessels, for example 2 milliliter cylindrical reaction vials having a diameter of about 10 millimeters, a large excess of the second reactant system is introduced simultaneously into each reaction vessel, and reaction between the two reactant systems in each of the vessels is allowed to proceed under identically controlled conditions of temperature and pressure for a single period of time. The reactions are simultaneously halted and the weight of the product relative to the weight of the original weight of the liquid comprising the first reactant system, the “weight percent of the product”, is measured for each vessel, using an analytical technique such as gas chromatography. Those reaction vessels in which the “weight percent of the product” is at a maximum indicate that in those reaction vessels the reaction rate of the homogeneous chemical reaction was essentially independent of the mass transport rate of the second reactant system into the liquid. It should be noted that at least two of the reaction vessels need to have achieved the maximum weight percent of the product in order to be confident that rate of reaction between the first reactant system and the second reactant system was essentially independent of the mass transport rate of the second reactant system into the liquid.

As noted, the liquid comprising the first reactant system may be arrayed in the reaction vessel as a film. Based on the discussion above, a film having a thickness approaching a monolayer should be optimal in terms of achieving the condition that rate of reaction between the first reactant system and the second reactant system be essentially independent of the mass transport rate of the second reactant system into the liquid. Apart from the technical challenges associated with depositing such a film in a typical reaction vessel, for example, a 2 milliliter reaction vial having a diameter of about 10 millimeters, the optimum film thickness may for other reasons, for example evaporation of the liquid, not equate to the thinnest possible film that can be formed in a given application.

An alternative means of determining the range of dimensions of the liquid which satisfy the requirement that the reaction rate of the homogeneous chemical reaction be essentially independent of the mass transport rate of the second reactant system into the liquid is provided below. For example, in a homogeneous liquid-phase reaction between gaseous oxygen and a first reactant system embodied in a liquid arrayed as a film in which the availability of oxygen in the liquid phase may be the rate limiting factor, mass transport can be evaluated in the following manner. First, it may be assumed that the reaction is a pseudo first order liquid-phase homogeneous reaction with respect to the potentially limiting gaseous reactant (dissolved in the liquid), oxygen, or is limited by this reaction. Second, oxygen mass transport effects in the gas phase may be ignored, since the transport rate in the gas phase is significantly higher than the transport rate in the liquid phase. Third, it may be assumed that the gas contacts the liquid film only on the top surface of the film and that the film has uniform thickness. It is noted that the amount of oxygen available at the gas-liquid interface can also be increased by increasing the pressure of the gas in the reaction vessel. With these assumptions, the steady state relationship among the liquid film thickness (L), the rate constant for the reaction (k), and the diffusivity (D) of dissolved oxygen in the liquid can be expressed as follows:

L=b\sqrt{D/k}

It is noted that k denotes a pseudo first order reaction rate constant of the homogeneous chemical reaction with respect to the dissolved form of the second reactant system, oxygen, in the liquid.

Although the diffusivity, D, and rate constant k are specific to individual reaction media and reactions respectively, methods for their determination are well known in the art. For example, methods for measuring the diffusivity, D, for a given gaseous reactant are well known in the art and are discussed in detail in A. H. P. Skelland, Diffusion Mass Transfer, Krieger Publishing Company, which is incorporated herein by reference. Additionally, there exist many compilations listing diffusivites, D, for gases in liquids. For example in the CRC Handbook of Chemistry and Physics, Robert C. Weast ed., CRC Press (1973), see table entitled “Diffusivities of Gases in Liquids”, page 55, which is also incorporated herein by reference.

Likewise, methods for determining rate constants, k, for a given reaction are well known in the art and are discussed in detail in texts such as, for example, H. Scott Folger, Elements of Chemical Reaction Engineering, Prentice Hall (1992) which is herein incorporated by reference, and Perry's Chemical Engineers' Handbook, Seventh Edition, Don W. Green, ed., McGraw-Hill (1997), see the entirety of Section 7: “Reaction Kinetics” which is also incorporated herein by reference. The value of the coefficient b may be derived as described below and is in a range between 0 and 5, preferably between 0 and 2.

The rates themselves should be substantial enough to be accurately measurable, so that differences among rates can be evaluated, thus allowing comparison among potential reactants and catalysts. In this context, it is preferred that b has a value between 0 and 5. This defines a minimum average-to-surface dissolved oxygen concentration ratio (or reaction rate) of approximately 20% (b˜5). More preferably, b has a value between 0 and 2, which defines a minimum average-to-surface concentration ratio of approximately 48% (b˜2). In various applications, other acceptable values for b can be determined with reference to the following relationship between the film thickness and the concentration profile:

$$\frac{{C}_{A} ( z )}{{C}_{A0}} = \frac{cos h b ( 1 - z / L )}{cos h b}$$

In the preceding relationship, the value of z is 0 at one surface of the film (i.e., the surface in contact with the gas, (“top” surface)), and, if the reaction is carried out in a vessel that supports the film from the bottom, the value of z is L at the opposing surface of the film (i.e., the bottom). It is further noted that the film may be supported on its sides (e.g., in a capillary tube or the like) or may be suspended in another manner that allows gas to be presented to both the bottom and top surfaces of the film simultaneously. In this situation, the value of z is L at the midpoint of the film (only half of the film is considered, since the other half is a mirror image).

As noted, mass transport in the gaseous phase may be increased by pressurizing the gas (or continuously replenishing the gas), therefore, it is preferred that the gas be maintained at a pressure greater than 1 atm while in contact with the liquid. Many homogeneous reactions respond favorably to increased temperature; therefore, in alternative embodiments, the liquid can be maintained at temperatures above 0° C. while in contact with the gas.

An alternative embodiment of the present invention provides a method of performing simultaneous homogeneous chemical reactions utilizing multiphase reactant systems. The method includes the steps of providing a combinatorial micro-reactor comprising a first vessel and a second vessel; placing a first reactant system embodied in a first liquid into the first vessel; and placing a second reactant system embodied in a second liquid into the second vessel. The first liquid is contacted with a third reactant system embodied in a first gas, and the first liquid is arrayed in a form, for example a film, layer, droplet, bead, strand, ring, or like array, having dimensions such that the reaction rate of the homogeneous chemical reaction is essentially independent of the mass transport rate of the third reactant system into the first liquid. The second liquid is contacted with a fourth reactant system embodied in a second gas, and the second liquid is arrayed in a form such as a film, layer, droplet, bead, strand, ring, or like array, having dimensions such that the reaction rate of the homogeneous chemical reaction is essentially independent of the mass transport rate of the fourth reactant system into the second liquid. Additional vessels can be added to the combinatorial micro-reactor as needed.

This embodiment of the present invention is useful for rapid parallel screening of reactant system components. Accordingly, depending upon the purpose of the reactions, the first reactant system and the second reactant system can include identical compounds in the same or differing quantities. Likewise, the third reactant system and the fourth reactant system can include identical compounds in the same or differing quantities. Furthermore, the first liquid and the second liquid can be chemically identical, and the first gas and the second gas can be chemically identical. Those skilled in the art will realize that the present method can be used to isolate the effects of changes in the identity of reactant system components, component ratios, and reaction conditions in order to optimize a desired characteristic of a given reaction.

As noted, the present invention is also directed to an apparatus for rapid screening of multiphase reactant systems. An exemplary embodiment is shown in FIG. 1 in which a vessel 10 contains a first reactant system embodied in a liquid 12 and a second reactant system embodied in a gas 14. Liquid 12 is arrayed in the form of a film having a thickness L, the thickness L being such that the reaction rate of the resulting homogeneous chemical reaction is essentially independent of the mass transport rate of the second reactant system into liquid 12. Acceptable values for L can be readily determined by using the relationships discussed supra or by routine experimentation as noted.

Vessel 10 is preferably formed of a rigid material that is chemically inert in the reaction environment. An example of an acceptable vessel for many reactions is a glass or quartz vial, for example a 2 milliliter cylindrical glass or quartz vial having a diameter of about 10 millimeters. When dealing with liquids with high vapor pressures or with reactions requiring long reaction times, it may be desirable to provide a covering, such as a selectively permeable cap 16 or a septum (not shown) incorporating a feed tube or needle disposed on vessel 10 such that gas 14 is allowed to move freely into and out of vessel 10 while depletion of liquid 12 by evaporation is minimized. This arrangement allows an external pressure source to act upon gas 14 while limiting the evaporation of liquid 12. In most applications, suitable materials for the cap include polytetrafluoroethylene (PTFE) and expanded PTFE. A suitable cap for use with 2 ml glass vials is “Clear Snap Cap, PTFE/Silicone/PTFE with Starburst, 11 mm”, part no. 27428, available from Supelco, Inc., Bellefonte, Pa.

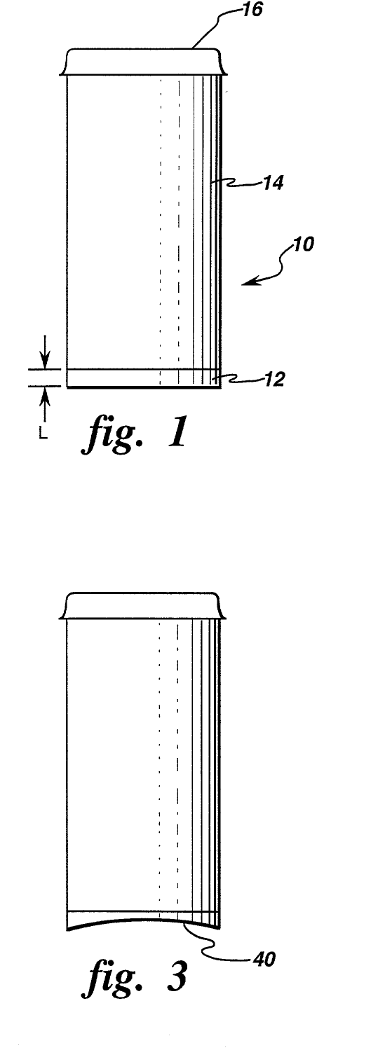

As shown in FIG. 2, the present invention is also directed to a combinatorial micro-reactor comprising a first vessel 10 and a second vessel 20. First vessel 10 contains a first reactant system embodied in a first liquid 12 and a second reactant system embodied in a first gas 14. First liquid 12 is arrayed in the form of a film having a thickness L, the thickness L being such that the reaction rate of the homogeneous chemical reaction is essentially independent of the mass transport rate of the second reactant system into first liquid 12. Second vessel 20 contains a third reactant system embodied in a second liquid 22 and a fourth reactant system embodied in a second gas 24. Second liquid 22 is arrayed in the form of a film having a thickness L, the thickness L being such that the reaction rate of the homogeneous chemical reaction is essentially independent of the mass transport rate of the fourth reactant system into second liquid 22.

The combinatorial micro-reactor can further include a substrate 36 having a plurality of discrete wells 38 adapted to receive vessels 10, 20 therein. Substrate 36 can be formed of any material capable of supporting and separating vessels 10, 20 provided that the material does not affect the reactions. In various applications, desired reaction conditions can include elevated temperatures within liquid 12, 22. In these circumstances it may be desirable to form substrate 36 of a thermally conductive material so that temperature within the liquid can be more easily controlled with an external device. In applications that require elevated temperatures and pressures, substrate 36 can be placed in an autoclave (not shown) or other device capable of maintaining these reaction conditions in preferred ranges. If additional capacity is needed, multiple vessels can be inserted into each well by linearly stacking the vessels.

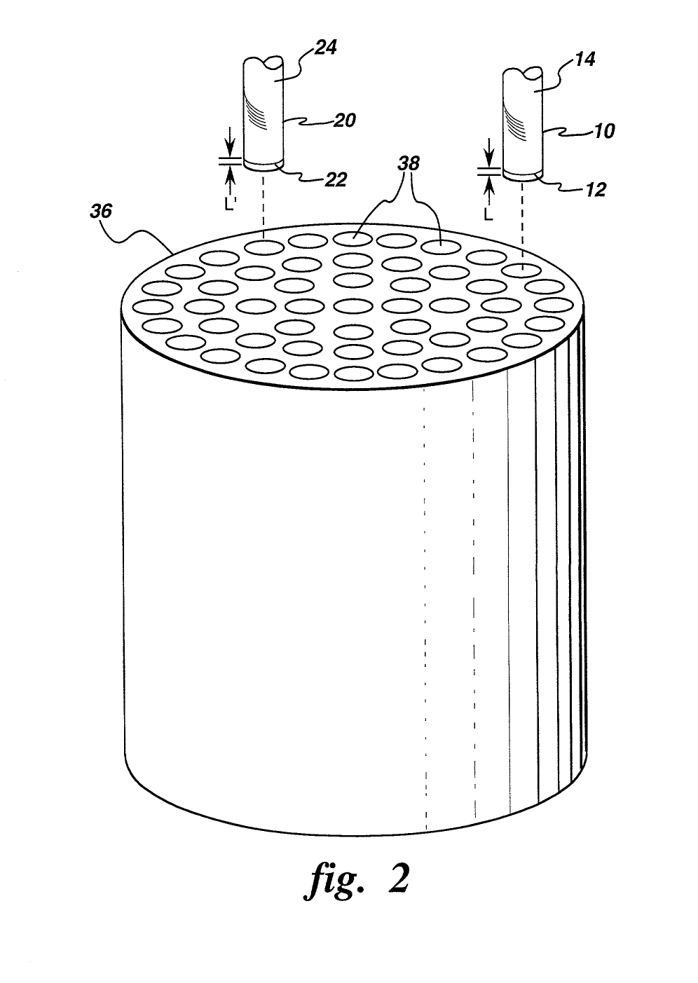

In order that the liquid may be arrayed in a form having substantially uniform dimensions, it is preferable to utilize vessels with substantially planar bottom sections, such as those depicted in FIG. 1 and FIG. 2. However, most commercially available small vials are geometrically similar to the vial shown in FIG. 3, where the bottom section 40 is concave. It is noted that the method of the present invention can be performed in such a vial and that the teachings of the present application provide guidance in choosing workable ranges for film thickness when employing these reaction vessels.

### EXAMPLES

The following examples are provided in order that those skilled in the art will be better able to understand and practice the present invention. These examples are intended to serve as illustrations and not as limitations of the present invention as defined in the claims herein.

Diphenyl carbonate (DPC) is useful, inter alia, as an intermediate in the preparation of polycarbonates. One method for producing DPC involves the carbonylation of a hydroxyaromatic compound (e.g., phenol) in the presence of a catalyst system. A carbonylation catalyst system typically includes a Group VIII B metal (e.g., palladium), a halide composition, and a combination of inorganic co-catalysts (IOCCs). This one step reaction is typically carried out in a continuous reactor at high temperature and pressure with gas sparging. Insufficient gas/liquid mixing can result in low yields of DPC. Generally, testing of new catalyst systems has been accomplished at macro-scale and, because the mechanism of this carbonylation reaction is not fully understood, the identity of additional effective IOCCs has eluded practitioners. An embodiment of the present invention allows this homogeneous carbonylation reaction to be carried out in parallel with various potential catalyst systems and, consequently, this embodiment has been used to identify effective IOCCs for the carbonylation of phenol.

The economics of producing DPC by the carbonylation process is partially dependent on the number of moles of DPC produced per mole of Group Vm B metal utilized. In the following examples, the Group VIII B metal utilized is palladium. For convenience, the number of moles of DPC produced per mole of palladium utilized is referred to as the palladium turnover number (Pd TON).

The palladium turnover number (Pd TON) is used interchangeably with the “weight percent of the product” DPC. For example, in the Table in Example 1 the Palladium turnover number for Sample 1 of 3417 and the “weight percent of product” DPC of 13.8 percent are related as follows. The palladium concentration is 0.2 mM (0.2 mmole per liter). Thus, 24 μl (microliters) of the reactant system comprising the palladium catalyst contains 4.8×10⁻⁶ moles of the palladium catalyst. Following the reaction the reaction mixture was found to contain 13.8 percent by weight DPC. The weight of the liquid phase (density=1.06 mg/μl ) employed was 25.4 mg. Thus the reaction produced 3.5 mg (0.0164 mmole DPC). The calculated palladium turnover number (Pd TON) is thus 3417.

Unless otherwise specified, all parts are by weight; all equivalents are relative to palladium; and all reactions were carried out in 2 ml glass vials at 90-100° C in a 10% O₂ in CO atmosphere at an operating pressure of 95-110 atm. Reaction time was generally 2-3 hours. Reaction products were verified by gas chromatography.

### Example 1

A liquid reactant system was prepared by adding 1,4 -bis(diphenylphosphino)butane palladium(II) dichloride (“Pd(dppb)Cl₂”), 240 equivalents of bromide in the form of tetraethylammonium bromide (“TEAB”), 56 equivalents of lead in the form of lead(II) oxide, and 8 equivalents of cerium in the form of cerium (III) acetylacetonate to phenol. Assorted aliquots of the liquid reactant system were placed, at ambient conditions, in 2 ml glass vials. The vials were placed in individual wells in an aluminum substrate. The substrate was placed in an autoclave, where a 9% O₂ in CO atmosphere was introduced into the vials at a pressure of 109 atm. The liquid was heated to a temperature of 100° C. These reaction conditions were maintained for 3 hours. The substrate was then removed from the autoclave; the vials were removed from the substrate; and samples from each of the vials were analyzed to provide the following results:

| Sample | Pd(dppb)Cl₂ | Sample Size | DPC | |
|---|---|---|---|---|
| No. | (mM) | (μl) | (wt %) | Pd TON |
| 1 | 0.20 | 24 | 13.8 | 3417 |
| 2 | 0.20 | 27 | 14.1 | 3487 |
| 3 | 0.20 | 99 | 10.2 | 2528 |
| 4 | 0.20 | 101 | 13.0 | 3213 |
| 5 | 0.25 | 293 | 3.0 | 593 |
| 6 | 0.25 | 306 | 2.9 | 569 |

The data show that sample size (and consequently film thickness) affects the reaction yield. For the carbonylation of phenol with the catalyst system, reaction vial size, and other reaction conditions used, the data show that a sample size of about 25 μL is preferred.

### Example 2

In order to determine whether results obtained using the thin film micro-reactor effectively correlate with results obtained from a macro-scale reactor, tests were conducted in discrete reactors. One set of tests was performed in a thin film micro-reactor according to the method of Example 1. The other set of tests was preformed in a “batch-flow” reactor. The batch-flow reactor allows a liquid reaction mixture to be fed into a reaction chamber. The system is then sealed, and pressurized gaseous reactants are continuously introduced into and removed from the reaction chamber. The reaction chamber and the entering gaseous reactants are heated to a desired temperature. In addition to the agitation caused by the continuous introduction of the gaseous reactants, the liquid reaction mixture is constantly stirred to effect mixing of the phases and to minimize settling of any precipitate. Molecular sieves are disposed in the reaction chamber to function as desiccants. Aliquots of the reaction mixture can be periodically withdrawn and analyzed to monitor the reaction and to determine yield.

The correlation data was produced by reacting phenol with carbon monoxide in the presence of the following catalyst system: 0.25 mM palladium(II) acetylacetonate (“Pd(acac)₂”), 56 equivalents of PbO, various amounts of cerium(III) acetylacetonate (“Ce(acac)₃”) and various amounts of an organic bromide salt, either TEAB, tetramethylammonium bromide (“TMAB”), or hexaethylguanidinium bromide (“HegBr”). The reactions were carried out at 100° C. in a 10% O₂ in CO atmosphere. Product samples were obtained after 3 hours of reaction time and analyzed for Pd TON to produce the following data:

BATCH-FLOW REACTOR

| Sample | Ce(acac)₃ | TEAB | TMAB | |
|---|---|---|---|---|
| No. | Equivalents | Equivalents | Equivalents | Pd TON |
| 1 | 0 | 160 | 0 | 878 |
| 2 | 8 | 80 | 0 | 2230 |
| 3 | 8 | 80 | 0 | 1828 |
| 4 | 8 | 160 | 0 | 2921 |
| 5 | 8 | 330 | 0 | 4466 |
| 6 | 8 | 0 | 320 | 5411 |
| 7 | 16 | 0 | 320 | 4234 |

THIN FILM MICRO-REACTOR

| | Ce(acac)₃ | HegBr | |
|---|---|---|---|
| Sample No. | Equivalents | Equivalents | Pd TON |
| 1 | 0 | 150 | 300 |
| 2 | 0 | 150 | 472 |
| 3 | 2 | 150 | 2655 |
| 4 | 4 | 150 | 3147 |
| 5 | 8 | 150 | 3191 |
| 6 | 16 | 150 | 2765 |
| 7 | 8 | 60 | 1554 |

As can be seen above, the micro-reactor correctly identified 8 equivalents as the preferred amount of cerium for the same reaction in the batch-flow reactor. Furthermore, results from the micro-reactor correctly predict that, for the reaction conditions used, Pd TON will increase as bromide concentration increases. Although the Pd TONs at a given concentration are not identical between the two reactors, it is evident that the correlation between the performances of the two reactors allows for meaningful discrimination among potential reactants using the thin film micro-reactor.

The method of Example 1 was repeated with the combination of palladium(II) acetylacetonate, HegBr, and manganese(III) acetylacetonate as a catalyst system. The sample size for all samples was 25 μL. The vials were exposed to a 10% O₂ in CO atmosphere at 100° C. and 98 atm for 3 hours. The following results were observed:

| | Pd(acac)₂ | Mn(acac)₃ | HegBr | |
|---|---|---|---|---|
| Sample No. | mM | Equivalents | Equivalents | Pd TON |
| 1 | .25 | 2 | 10 | 104 |
| 2 | .25 | 2 | 600 | 423 |
| 3 | .25 | 6 | 150 | 440 |
| 4 | .25 | 20 | 10 | 294 |
| 5 | .20 | 20 | 600 | 40.6 |

### Example 4

The method of Examples 1 and 3 was repeated with palladium(II) acetylacetonate, HegBr, and copper(II) acetylacetonate as an inorganic co-catalyst. The reactants were heated to 100° C. for 3 hours in a 10% oxygen in carbon monoxide atmosphere. After the reaction, samples were analyzed for DPC by gas chromatography. The following results were observed:

| | Pd(acac)₂ | Cu(acac)₂ | HegBr | |
|---|---|---|---|---|
| Sample No. | mM | Equivalents | Equivalents | Pd TON |
| 1 | .25 | 28 | 120 | 1320 |
| 2 | .25 | 28 | 30 | 318 |
| 3 | .25 | 20 | 600 | 1067 |
| 4 | .25 | 20 | 10 | 216 |
| 5 | .20 | 17.5 | 750 | 1993 |
| 6 | .25 | 14 | 600 | 1850 |
| 7 | .25 | 14 | 120 | 1184 |
| 8 | .25 | 14 | 60 | 707 |
| 9 | .25 | 14 | 30 | 424 |
| 10 | .25 | 2 | 10 | 211 |

### Example 5

The method of Examples 1, 3, and 4 was repeated with 0.25 mM palladium(II) acetylacetonate, various amounts of bromide, and various amounts of manganese(III) acetylacetonate and bismuth(II) tetramethylhetptanedionate as IOCCs to provide the following results:

| | Mn(acac)₃ | Bi(TMHD)₂ | HegBr | |
|---|---|---|---|---|
| Sample No. | Equivalents | Equivalents | Equivalents | Pd TON |
| 1 | 14 | 2.8 | 120 | 645 |
| 2 | 14 | 2.8 | 30 | 583 |
| 3 | 28 | 5.6 | 120 | 728 |
| 4 | 28 | 5.6 | 30 | 564 |
| 5 | 2.8 | 14 | 120 | 818 |
| 6 | 2.8 | 14 | 30 | 477 |
| 7 | 5.6 | 28 | 120 | 1075 |
| 8 | 5.6 | 28 | 30 | 556 |

### Example 6

The method of Examples 1 and 3-5 was repeated with 0.25 mM palladium(II) acetylacetonate, various amounts of HegBr, and various amounts of the IOCC combination of iron(III) acetylacetonate and bismuth(II) tetramethylheptanedionate. The following results were observed:

| Experiment | Fe(acac)₃ | Bi(TMHD)₂ | HegBr | |
|---|---|---|---|---|
| No. | Equivalents | Equivalents | Equivalents | Pd TON |
| 1 | 2.8 | 14 | 120 | 372 |
| 2 | 2.8 | 14 | 30 | 216 |
| 3 | 5.6 | 28 | 120 | 368 |
| 4 | 5.6 | 28 | 30 | 231 |
| 5 | 14 | 2.8 | 120 | 208 |
| 6 | 14 | 2.8 | 30 | 474 |
| 7 | 28 | 5.6 | 120 | 377 |
| 8 | 28 | 5.6 | 30 | 732 |

Based on the results of these experiments, it is evident that the method and apparatus of the present invention can effectively discriminate among various reaction conditions in a homogeneous reaction utilizing multiphase reactants.

It will be understood that each of the elements described above, or two or more together, may also find utility in applications differing from the types described herein. While the invention has been illustrated and described as embodied in a method apparatus for rapid screening of multiphase reactant systems, it is not intended to be limited to the details shown, since various modifications and substitutions can be made without departing in any way from the spirit of the present invention. For example, robotic equipment can be used to prepare the samples and various types of parallel screening methods can be incorporated. As such, further modifications and equivalents of the invention herein disclosed may occur to persons skilled in the art using no more than routine experimentation, and all such modifications and equivalents are believed to be within the spirit and scope of the invention as defined by the following claims.

What is claimed is:

1. A method of performing a homogeneous chemical reaction utilizing multiphase reactant systems, said method comprising the steps of:
providing a first reactant system embodied in a liquid;
contacting the liquid with a second reactant system embodied in a gas, the second reactant system having a mass transport rate into the liquid;
wherein the liquid is arrayed in a form having dimensions such that the reaction rate of the homogeneous chemical reaction is essentially independent of the mass transport rate of the second reactant system into the liquid.

2. The method of claim 1, wherein the gas is maintained at a pressure greater than 1 atm while in contact with the liquid.

3. The method of claim 1, wherein the liquid is maintained at a temperature above 0° C. while in contact with the gas.

4. The method of claim 1, wherein the liquid is a component of the first reactant system.

5. The method of claim 4, wherein the first reactant system comprises a hydroxyaromatic compound.

6. The method of claim 1, wherein the gas is a component of the second reactant system.

7. The method of claim 6, wherein the second reactant system comprises carbon monoxide.

8. The method of claim 1, wherein the second reactant system is dissolved in the gas.

9. The method of claim 1, wherein the first reactant system comprises a catalyst system.

10. The method of claim 9, wherein the catalyst system comprises a Group VIII B metal.

11. The method of claim 10, wherein the Group VIII B metal is palladium.

12. The method of claim 10, wherein the catalyst system includes a halide composition.

13. The method of claim 10, wherein the catalyst system includes an inorganic co-catalyst.

14. The method of claim 13, wherein the catalyst system includes a combination of inorganic co-catalysts.

15. The method of claim 1, further comprising the step of limiting the evaporation of the liquid while permitting the gas to contact the liquid.

16. A method of performing simultaneous homogeneous chemical reactions utilizing multiphase reactant systems, said method comprising the steps of:
providing a combinatorial micro-reactor comprising a first vessel and a second vessel;
placing a first reactant system embodied in a first liquid into the first vessel;
placing a second reactant system embodied in a second liquid into the second vessel;
contacting the first liquid with a third reactant system embodied in a first gas, the third reactant system having a mass transport rate into the first liquid;
wherein the first liquid is arrayed in a form having dimensions such that the reaction rate of the homogeneous chemical reaction is essentially independent of the mass transport rate of the third reactant system into the first liquid;
contacting the second liquid with a fourth reactant system embodied in a second gas, the fourth reactant system having a mass transport rate into the second liquid;
wherein the second liquid is arrayed in a form having dimensions such that the reaction rate of the homogeneous chemical reaction is essentially independent of the mass transport rate of the fourth reactant system into the second liquid.

17. The method of claim 16, wherein the first reactant system and the second reactant system comprise the same compound.

18. The method of claim 16, wherein the third reactant system and the fourth reactant system comprise the same compound.

19. The method of claim 16, wherein the first liquid and the second liquid are chemically identical.

20. The method of claim 16, wherein the first gas and the second gas are chemically identical.

21. A method of producing a homogeneous chemical reaction utilizing multiphase reactant systems, said method comprising the steps of:
providing a first reactant system embodied in a liquid;
contacting the liquid with a second reactant system embodied in a gas;
wherein the liquid is arrayed in the form of a film having a thickness L, said thickness L satisfying the following relationship:
L=b\sqrt{D/k}
wherein
L denotes the film thickness,
D denotes the diffusivity of the second reactant system in the liquid,
k denotes a pseudo first order reaction constant of the homogeneous chemical reaction with respect to the dissolved form of the second reactant system in the liquid, and
b has a value between 0 and 5.

22. The method of claim 21, wherein the gas is maintained at a pressure greater than 1 atm while in contact with the liquid.

23. The method of claim 21, wherein the liquid is maintained at a temperature above 0° C. while in contact with the gas.

24. The method of claim 21, wherein the liquid is a component of the first reactant system.

25. The method of claim 24, wherein the first reactant system comprises a hydroxyaromatic compound.

26. The method of claim 21, wherein the gas is a component of the second reactant system.

27. The method of claim 26, wherein the second reactant system comprises carbon monoxide.

28. The method of claim 21, wherein the second reactant system is dissolved in the gas.

29. The method of claim 21, wherein the first reactant system comprises a catalyst system.

30. The method of claim 29, wherein the catalyst system comprises a Group VIII B metal.

31. The method of claim 30, wherein the Group VIII B metal is palladium.

32. The method of claim 30, wherein the catalyst system includes a halide composition.

33. The method of claim 30, wherein the catalyst system includes an inorganic co-catalyst.

34. The method of claim 33, wherein the catalyst system includes a combination of inorganic co-catalysts.

35. The method of claim 21, further comprising the step of limiting the evaporation of the liquid while permitting the gas to contact the liquid.

36. The method of claim 21, wherein b has a value between 0 and 2.

37. A vessel containing a first reactant system embodied in a liquid and a second reactant system embodied in a gas, the second reactant system having a mass transport rate into the liquid, wherein the liquid is arrayed in a form having dimensions such that the reaction rate of the resulting homogeneous chemical reaction is essentially independent of the mass transport rate of the second reactant system into the liquid.

38. The vessel of claim 37, wherein the liquid is a component of the first reactant system.

39. The vessel of claim 38, wherein the first reactant system comprises a hydroxyaromatic compound.

40. The vessel of claim 37, wherein the gas is a component of the second reactant system.

41. The vessel of claim 40, wherein the second reactant system comprises carbon monoxide.

42. The vessel of claim 37, wherein the second reactant system is dissolved in the gas.

43. The vessel of claim 37, wherein the first reactant system comprises a catalyst system.

44. The vessel of claim 43, wherein the catalyst system comprises a Group VIII B metal.

45. The vessel of claim 44, wherein the Group VIII B metal is palladium.

46. The vessel of claim 44, wherein the catalyst system includes a halide composition.

47. The vessel of claim 44, wherein the catalyst system includes an inorganic co-catalyst.

48. The vessel of claim 47, wherein the catalyst system includes a combination of inorganic co-catalysts.

49. The vessel of claim 37, further comprising a selectively permeable cap disposed on the vessel such that gas is allowed to move freely into and out of the vessel while depletion of the liquid by evaporation is minimized.

50. A combinatorial micro-reactor comprising a first vessel and a second vessel,
the first vessel containing a first reactant system embodied in a first liquid and a second reactant system embodied in a first gas, the second reactant system having a mass transport rate into the first liquid, wherein the first liquid is arrayed in a form having dimensions such that the reaction rate of the homogeneous chemical reaction is essentially independent of the mass transport rate of the second reactant system into the first liquid;
the second vessel containing a third reactant system embodied in a second liquid and a fourth reactant system embodied in a second gas, the fourth reactant system having a mass transport rate into the second liquid, wherein the second liquid is arrayed in a form such that the reaction rate of the homogeneous chemical reaction is essentially independent of the mass transport rate of the fourth reactant system into the second liquid.

51. The combinatorial micro-reactor of claim 50, wherein the first reactant system and the third reactant system comprise the same compound.

52. The combinatorial micro-reactor of claim 50, wherein the second reactant system and the fourth reactant system comprise the same compound.

53. The combinatorial micro-reactor of claim 50, wherein the first liquid and the second liquid are chemically identical.

54. The combinatorial micro-reactor of claim 50, wherein the first gas and the second gas are chemically identical.

55. The combinatorial micro-reactor of claim 50, further comprising a substrate having a plurality of discrete wells adapted to receive the vessels therein.

56. The combinatorial micro-reactor of claim 55, further comprising an autoclave adapted to receive the substrate.

57. The combinatorial micro-reactor of claim 50, further comprising a selectively permeable cap disposed on each vessel such that gas is allowed to move freely into and out of the vessel while depletion of the liquid by evaporation is minimized.

58. A vessel for accommodating a homogeneous chemical reaction, said vessel containing a first reactant system embodied in a liquid and a second reactant system embodied in a gas, wherein the liquid is arrayed in the form of a film having a thickness L, said thickness L satisfying the following relationship:
L=b\sqrt{D/k}
wherein
L denotes the film thickness,
D denotes the diffusivity of the second reactant system in the liquid,
k denotes a pseudo first order reaction constant of the homogeneous chemical reaction with respect to the dissolved form of the second reactant system in the liquid, and
b has a value between 0 and 5.

59. The vessel of claim 58, wherein b has a value between 0 and 2.

60. The vessel of claim 58, wherein the liquid is a component of the first reactant system.

61. The vessel of claim 60, wherein the first reactant system comprises a hydroxyaromatic compound.

62. The vessel of claim 58, wherein the gas is a component of the second reactant system.

63. The vessel of claim 62, wherein the second reactant system comprises carbon monoxide.

64. The vessel of claim 58, wherein the second reactant system is dissolved in the gas.

65. The vessel of claim 58, wherein the first reactant system comprises a catalyst system.

66. The vessel of claim 65, wherein the catalyst system comprises a Group VIII B metal.

67. The vessel of claim 66, wherein the Group VIII B metal is palladium.

68. The vessel of claim 66, wherein the catalyst system includes a halide composition.

69. The vessel of claim 66, wherein the catalyst system includes an inorganic co-catalyst.

70. The vessel of claim 69, wherein the catalyst system includes a combination of inorganic co-catalysts.
